(19) United States
(12) Patent Application Publication
(10) Pub. No.: US 2004/0071782 A1
(43) Pub. Date: Apr. 15, 2004

(54) Ionophore antibiotic formulations

(76) Inventors: Kim Ewing Melville Agnew, Auckland (NZ); William Austin Hewitt, Auckland (NZ); Craig Richard Hanham, Auckland (NZ); Kevin Grant Purdy, Auckland (NZ)

Correspondence Address: Kirby W Lee, Eli Lilly and Company, Patent Division, P O Box 6288, Indianapolis, IN, 46206-6288, US

(21) Appl. No.: 10/450,079

(22) PCT Filed: Dec. 18, 2001
(86) PCT No.: PCT/NZ01/00290

(30) Foreign Application Priority Data
Dec. 19, 2000 (NZ) 509092
Sep. 13, 2001 (NZ) 514183

(51) Int. Cl. A61K 31/704; A61K 9/14

(52) U.S. Cl. 424/489; 514/37

(57) ABSTRACT

The invention relates to an ionophore antibiotic composition capable of dilution with water to a substantially stable dispersed form in all water then present, said composition comprising or including:—at least one ionophore antibiotic (preferably monensin) of a mean particle size of less than 20 microns,—and at least one dispersing agent. A method of preparing the ionophore antibiotic composition is also disclosed.

The present invention relates to ionophore antibiotic compositions and particularly, but not solely, to ionophore antibiotic compositions capable of dilution with water, suitable for inclusion directly or via a holding tank in the drinking water of an animal to have administered and/or self administered an ionophore antibiotic or ionophore antibiotics.

### BACKGROUND

Administration of ionophore antibiotics such as monensin to animals (preferably ruminants) is known to achieve in appropriate dosages advantages for a number of different purposes. These include the treatment or prevention of ketosis and/or bloat, the enhancement of milk production, enhancement of milk protein content in milk, enhancement of mineral uptake, enhancement of weight gain, and/or enhancement of feed conversion efficiency (in ruminants), desirable reproduction advantages and as a milk replacement. See U.S. Pat. No. 3,829,557.

In this respect we refer to European Patent Specification 0,139,595 A2 of KOFFOLK (1949) LTD which relates to a liquid ionophore antibiotic composition for ruminants and poultry where the antibiotic is dissolved in a non-toxic water-soluble organic solvent rather than a water soluble organic solvent, and in use the resulting solution is admixed with a liquid feed, a liquid vitamin concentrate or drinking water. There are claims for stability on standing.

The composition of EP 0,139,595 indicates that because monensin and its sodium salt is only slightly soluble in water that it is generally administered in a dry form in an animal feed and/or in dry liquid milk replacer compositions. The same is also indicated as being true for another ionophore antibiotic lasalocid which in U.S. Pat. No. 3,715,372 is reported to be completely insoluble in water.

The composition of EP 0,139,595 uses as an organic solvent for the ionophore antibiotic a solvent selected from the group comprising propylene glycol, glycerol, ethanol and isopropanol and mixtures thereof.

Example 1 of EP 0,139,595 indicates that 250 g of a mycelium containing 10% monensin was mixed at room temperature with 1250 cc propylene glycol for 2 hours by exposing the mixture to ultrasound. There is an indication that 50% of the monensin was found in solution in the propylene glycol.

Monensin is usually available commercially as the sodium salt of the acid. Monensin sodium is available in two forms, namely, a crystalline form or a mycelial form. The mycelial form has only about a 20% activity while the crystalline form has greater than 90% active monensin sodium.

Reference herein to “monensin” where the context allows encompasses all forms thereof including monensin, alkali metal salts of monensin and monensin esters and includes mixtures. Likewise for the other ionophore antibiotics.

In our New Zealand Patent Specification 272574/272940 (equivalent to PCT/NZ96/00068 WO 97/03650) we disclose an aqueous base suspension concentrate of an ionophore antibiotic or ionophore antibiotics such as monensin.

In one aspect in NZ 272574/272940 that invention is defined as an aqueous base suspension concentrate of an ionophore antibiotic or ionophore antibiotics capable of aqueous dilution (if desired) and capable (with or without such aqueous dilution) of being orally administered to an animal by an active dosing regime (eg; by drenching), said concentrate comprising or including

(I) at least one ionophore antibiotic in

(II) an aqueous system containing

(i) a wetting and/or surfactant agent, (preferably alkyl polyglycoside)

(ii) an antifreeze agent or agents in which the ionophore antibiotic or antibiotics is or are no more than sparingly soluble,

(iii) a suspension agent (eg gum(s)),

(iv) optionally, an antifoam agent or system,

(v) optionally, a preservative,

(vi) optionally, a de-bittering agent,

(vii) optionally, a pH buffering system, and

(viii) water.

The preferred ionophore antibiotic of NZ 272574/272940 is sodium monensin. Preferably an antifreeze agent or agents in which the ionophore antibiotic(s) is no more than sparingly soluble (such as a glycol or polyglycol) is present. Additional, preferably an antifoam agent is present (e.g. simethicone and a particulate carrier such as silicon dioxide therefor).

NZ 272574/272940 also discloses a method of drenching an animal with such an ionophore antibiotic which involves administering orally to such an animal a diluted form of the compositions, such dilution being with water.

We have found monopropylene glycol to be a poor solvent of monensin. While we have in NZ 272574/272940 referred to monopropylene glycol as being an antifreeze agent in which the ionophore antibiotic is sparingly soluble, we do appreciate that solubility to the extent referred to in Example 1 of EP 0,139,595 may be achieved when induced with ultrasound or heating.

The form of crystalline monensin used for the examples in NZ272574/272940 has a mean particle size of the order of about 40 microns and in order to hold such large particles in suspension an aggressive supporting system for that purpose has been required.

Thus despite the advances represented by the invention disclosed in NZ272574/272940 (providing compositions useful for an active system of administering ionophore antibiotics to ruminants), and despite assertions of EP139595, there continues to be a substantial need for compositions containing ionophore antibiotics which are inherently poorly soluble or insoluble in aqueous based systems, suitable for passive system administrations to ruminants. Such compositions must, to avoid under-dosing or toxic dosing, remain substantially homogeneous for extended periods of time.

The present invention investigates alternatives to such aggressive systems and targets a dosable composition capable of dispersion stability as a suspension in highly dilute drinking water for animals.

The present invention relies upon both a preferred smaller mean particle size of the ionophore antibiotic and an associated regime including related methods of manufacture, uses etc.

The present invention also recognises inter alia the therapeutic, energy and productivity benefits were monensin or any other suitable ionophore antibiotic (see the listing in our aforementioned patent specification, and below) to be used as a trough treatment.

Trough Treatment Systems

Currently three types of trough treatment system for animal drinking water and not hitherto used with an ionophore antibiotic are known.

Three types are in use:

1. Proportional feed systems e.g. that branded DOSATRON™

delivers a %mix in proportion to demand (from 0.2-2% for dosatron). Aim is to spread delivery of the complete batch over 24 hours

2. Dump systems

Continually delivers drench into lines regardless of demand. Tends to be all used up within 5 hours.

3. Individual trough systems e.g. PETA™ dispenser

Delivers concentrated drench in relation to demand but pay out is skewed early on.

The present invention includes an ionophore antibiotic base composition capable of aqueous dilution which may be useful in such a trough treatment system. The object of the present invention is to provide such a composition and/or to provide drinking water for an animal to allow administration of an ionophore antibiotic to the animal.

We have determined that a microfine form of an ionophore antibiotic with its smaller mass to surface area ratio is a significant advantage over less fine (eg; 40 micron mean particle size) ionophore antibiotic inclusions in an aqueous composition and more so when it is to be used in such a way where it is subjected to unsupervised dilution, eg; to provide an infeed (directly or indirectly) into a dilution quantity of trough make up water. Such advantages to uniform suspendability we believe to be equally applicable to the more preferred crystalline forms as well as the less preferred mycellial forms of the ionophore(s).

We have also determined that a suitable glycol such as monopropylene glycol can be used in such a way to pre-prepare an ionophore antibiotic for subsequent suspension as a aqueous concentrate and thereafter to carry at least an antifreeze advantage over to its subsequent use (eg; as an infeed aqueous concentrate for a dose-certain water system) without leading to crashing out of the ionophore antibiotic. Glycols (such as monopropylene glycol) unless agressively treated provide no significant uptake of the stably suspended ionophore antibiotic (such as monensin) from an aqueous system. This we consider desirable since an organic uptake can lead to crashing out on dilution. For example, an organic solvent, such as methanol, allows monensin to crash out of solution if any water is added.

In the preferred forms of the present invention the ionophore antibiotic of choice is monensin in any of its appropriate forms (eg; sodium monensin). Other ionophore antibiotics however fall within the scope of the present invention and these include Lonomycin, Ionomycin, Laidlomycin, Nigericin, Grisorixin, Dianemycin, Lenoremycin, Salinomycin, Narasin, Antibiotic X206, Alborixin, Septamycin, Antibiotic A204, Maduramicin and Semduramicin, Compound 47224, Lasalocid (also including factors A, B, C, D and E), Mutalomycin, Isolasalocid A, Lysocellin, Tetronasin, Echeromycin, Antibiotic X-14766a, Antibiotic A23187, Antibiotic A32887, Compound 51532 and K41.

### STATEMENTS OF THE INVENTION

In a first aspect the present invention consists in a method of forming an aqueous suspension capable of subsequent dilution, said method including prior to any substantial presence of water and/or a suspension agent (such as a suitable gum), milling the ionophore antibiotic or ionophore antibiotics (preferably in a crystalline form) with at least a suitable glycol.

Preferably said milling is to a mean particle size very much less than 40 microns.

Preferably the mean particle size is less than 20 microns, more preferably less than 5 microns and most preferably into a mean particle size of from 0.1 to 1.0 microns.

Preferably said milling includes the presence of a suitable lignosulfonate and/or a suitable polyglycoside.

Preferably said milling includes the presence of an antifoam agent.

Preferably said milling includes at least some water.

Preferably no suspension agent (eg; a gum typified by xanthan gum or guar gum) is present at said milling.

Preferably said method is performed substantially as hereinafter described with reference to the accompanying drawing.

Preferably the product includes as its suitable glycol monopropylene glycol.

Preferably the aqueous suspension is of a kind hereinafter described which includes monopropylene glycol, an additional wetting agent (eg; a lignosulfonate or polyglycoside or both) and a suspension agent.

Preferably the ionophore antibiotic is monensin and preferably that monensin is in a crystalline form.

In a further aspect the present invention consists in a method of forming an aqueous suspension of at least one ionophore antibiotic capable of subsequent dilution, said method including milling the ionophore antibiotic or ionophore antibiotics (preferably in a crystalline form) with at least a suitable polyglycoside or a suitable lignosulfonate.

Preferably said method performs part of a method as previously defined.

In yet a further aspect the present invention consists in a method of forming an aqueous suspension of at least one ionophore antibiotic capable of subsequent dilution, said method comprising or including

milling an ionophore antibiotic or ionophore antibiotics (preferably in a crystalline form) with at least (i) a suitable glycol and (ii) at least one of a suitable lignosulfonate and a suitable polyglycoside, and

subsequently formulating the suspension with at least water or, optionally, additional water.

Preferably said subsequent formulation involves the addition of a suitable dispersion agent.

Preferably said suitable dispersion agent is a suitable gum typified by xanthan gum and guar gum.

Other suitable dispersion agents include hydroxy-ethyl cellulose, bentonite clay, montrnorillinite clay and fumed silica.

Preferably said ionophore antibiotic is of sufficient purity as to minimise any requirement for an anti foaming agent but if an anti foaming agent is necessary, preferably said anti foaming agent is a GENSIL™ system, i.e. of simethicone/silicon dioxide support for the simethicone.

Preferably said suspension includes a buffer system.

Preferably said suspension includes a preservative.

Where reference is made to a suitable glycol preferably said glycol is a liquid and preferably said glycol assumes an anti freezing role in the resultant aqueous suspension.

Example of a suitable glycol is monopropylene glycol but other examples include ethylene glycol, diethylene glycol and dipropylene glycol.

Preferably the ionophore antibiotic or ionophore antibiotics is at least primarily of a crystalline form but in other forms it can in part be of the mycelial form. For example, if as is preferred, the ionophore antibiotic is monensin (e.g. present for example as sodium monensin), preferably the ionophore antibiotic is substantially free of the mycelial form. Were the mycelial formed to be utilised however, preferably there are commensurate changes in the inclusions to reflect the lesser activity of that form or an additional input to any dilution system to achieve appropriate activities.

Preferably said milling is in liquid supported conditions (preferably as a result of a liquid glycol inclusion), preferably with a minimum of water necessary for the purpose (if any), and preferably is such as to reduce the antibiotic to a microfine form.

In a preferred form of the present invention, irrespective of whether or not the milling is dry or in a liquid environment, preferably the ionophore antibiotic is reduced to a mean particle size less than 50 microns.

Preferably said mean particle size is reduced to less than 20 microns.

Most preferably the microfine condition of the ionophore antibiotic is to a mean particle size less than 5 microns.

Most preferably the mean particle size resulting from the milling procedure or procedures (can be a single or multiple milling procedures) is such as to provide a mean particle size of the preferably crystalline ionophore antibiotic (e.g. monensin) in a range of from 0.1 to 1.0 microns.

Preferably the milling stage or stages involves milling with a suitable polyglycoside or lignosulfonate, or both. One such polyglycoside is alkyl polyglycoside. One possible lignosulfonate compound is ULTRAZINE NA™ or BORRESPERSE NA™ [of Borregaard Industries Ltd of Norway] (most preferably ULTRAZINE NA™). Optionally GENSIL™ or another suitable antifoam agent is also milled with the crystalline monensin or other antibiotic.

Preferably the milling of all coating agents is simultaneous although it can be in part or totally serially.

In a preferred mix the ionophore antibiotic is milled simultaneously with liquid monopropylene glycol and a suitable polyglycoside and/or lignosulfonate compound (more preferably a lignosulfonate) having a “wetting” function and (optionally) an antifoam agent and/or some water.

In a further aspect the present invention consists in an aqueous suspension formed by one or more of the methods of the present invention.

In still a further aspect the present invention consists in an aqueous ionophore antibiotic suspension capable of further dilution without any substantial crashing out of the ionophore inclusion, said aqueous concentrate comprising or including

an ionophore antibiotic,

monoproplyene glycol (or other suitable glycol),

optionally a further material having a wetting characteristic,

a suspension agent,

optionally an antifoam agent, and water,

wherein at least the monopropylene glycol and the ionophore antibiotic have been milled together prior to mixing with the water or at least any substantial amount of the water.

Preferably the further material having a wetting characteristic is a suitable polyglycoside or lignosulfonate.

Preferably said suitable polyglycoside is alkyl polyglycoside although more preferably the additional wetting agent is a lignosulfonate typified by ULLTRAZINE NA™ or BORRESPERSE NA™ (more preferably ULIRAZINE NA™).

Preferably the ionophore antibiotic is a crystalline form rather than a mycellial form.

Preferably the ionophore antibiotic is monensin or a monensin.

Preferably the ionophore antibiotic at least post milling has a particle size less than 5 microns and more preferably below 2 microns.

Preferably the particle size range of the antibiotic is from 0.1 microns to 1.0 microns.

Preferably the aqueous ionophore antibiotic suspension remains substantially homogeneous for a period longer than 7 days; more preferably for a period longer than 24 days.

It does not matter whether or not the milling reduces the ionophore antibiotic to the requisite mean particle size or whether or not it is already milled almost to that size. What is important is to have resultant particles of that size appropriately coated with the “wetting” agents which includes preferably a multifunctional glycol (eg; monopropylene glycol) and preferably an additional wetting agent.

In a further aspect the present invention consists in an aqueous ionophore antibiotic suspension comprising or including

0 to 20% w/v of a microfine (i.e. less than 40 micron mean particle size) crystalline ionophore antibiotic,

2 to 20% w/v of monopropylene glycol,

0 to 10% w/v of a wetting agent,

0.1 to 5% w/v suspension agent, and

water.

Preferably the aqueous ionophore antibiotic suspension remains substantially homogeneous for a period longer than 7 days; more preferably for a period longer than 24 days.

Preferably the formulation is substantially as described in any one of Examples 1 to 5.

In another aspect the present invention is directed to a composition suitable for providing a direct in feed and/or indirect in feed (e.g. via a make up tank) into a water system from which target animal can drink,

said composition being an aqueous composition of (at least)

(a) microfine ionophore antibiotic or microfine ionophore antibiotics,

(b) a glycol in which the ionophore antibiotic(s) is or are no more than sparingly soluble,

(c) a wetting agent,

(d) a suspension agent, and

(e) water, and

optionally any one or more of

an antifoam agent or system,

a preservative,

a debittering agent, and

a pH buffering system.

Preferably the aqueous ionophore antibiotic suspension remains substantially homogeneous for a period longer than 7 days; more preferably for a period longer than 24 days.

Preferably said suspension agent is xanthan gum.

Preferably said wetting and/or surfactant agent (c) is a lignosulphonate (e.g. ULTRAZINE NA™N) or a polyglycoside (preferably alkyl polyglycoside).

Preferably said ionophore antibiotic is in either a crystalline or mycellial form or both.

Preferably a crystalline form is utilised.

Preferably said ionophore antibiotic is monensin (e.g. sodium monensin).

Preferably said microfine ionophore antibiotic is of a particle size less than 5 microns (preferably less than 2 microns) and preferably has a mean particle size in the range of from 0.1 to 1.0 microns.

Preferably xanthan gum is present and in a quantity greater than 0.16 w/v % and preferably about 0.4 w/v % (ie, the amount required being more for the more concentrated aqueous concentrate, eg; will unlimited dilute none may be required).

Preferably the mode of mixing and formulation is substantially as disclosed herein with a preferred composition being substantially as follows (wherein preferably the mean sodium monensin particle size is substantially 5 microns)—

| Ingredient | | | |
|---|---|---|---|
| (common/chemical name) | Quantity (% w/w) | | Function |
| Sodium Monensin | 6.33% (about 6% monensin) w/v | | Ionophore antibiotic |
| Monopropylene glycol | 10% | w/v | Antifreeze/ |
| Alkyl Polyglycoside or | 0.5% | w/v | Surfactant/Wetting agent |
| ULTRAZINE NA ™ | 4-5% | w/v | |
| Disodium Phosphate Anhydrous | 0.355% | w/v | Buffer |
| MonoPotassium phosphate Dihydrate | 0.04% | w/v | Buffer |
| Dialkyl dimethyl ammonium bromide | .0064% | w/v | Preservative |
| Xanthan Gum | 0.4% | w/v | Suspension agent |
| Simethicone | 0.333% | w/v) | (e.g. GENSIL ™ |
| Silicon Dioxide | 0.167% | w/v) | Antifoam system) |

| | |
|---|---|
| Water | to 100% |

An alternative preferred formulation is (wherein preferably the mean sodium monensin particle size is substantially 5 microns):

| Ingredient | | |
|---|---|---|
| name (common or chemical) | Quantity (% w/w) | Function |
| Sodium Monensin QA 166H | 6.747% | Active |
| Monopropylene glycol | 10% | Antifreeze |
| Didecyl dimethyl Ammonium | 0.1% | Preservative |
| Bromide | | |
| Xanthan Gum | 0.5% | Suspension agent |
| Sodium lignosulphonate | 4.1% | Wetting agent |
| Alkyl Polyglycoside | 3.8% | Wetting agent |
| Simethicone | 0.333% | Antifoam |
| Silicon Dioxide | 0.167% | Antifoam |
| Water balance q.v | 74.47% | diluent |

In other aspect the present invention is a pre mill mix or post mill mix of the present invention as hereinafter described.

In yet a further aspect the present invention consists in a composition for inclusion in a water trough fed in system being a composition as previously defined which without dilution is adapted to be in feed into the water supply.

In still a further aspect the present invention consists in a drinking water supply for an animal (preferably a ruminant animal) which has an in feed therein of a composition as previously defined.

In yet a further aspect the present invention consists in a method of providing an ionophore antibiotic to a target mammal which comprises providing to the animal drinking water with a dispersed ionophore antibiotic, said ionophore antibiotic having been included in the water supply by an intake from a composition as previously defined.

According to a further aspect of the invention there is provided an ionophore antibiotic composition capable of dilution with water to a substantially stable dispersed form in all water then present, said composition comprising or including:

at least one ionophore antibiotic of a mean particle size of less than 20 microns,

and at least one dispersing agent.

Preferably the mean particle size of at least one ionophore antibiotic is substantially 5 microns.

Preferably the dispensed form in water remains substantially homogeneous for at least 24 days.

Preferably the at least one dispersing agent is selected from one or more of the following:

i) a compatible polyglycoside capable of acting as a dispersing agent,

ii) a compatible lignosulfonate capable of acting as a dispersing agent, and

iii) a compatible and suitable glycol in a form capable of acting as a dispersing agent.

Preferably a liquid vehicle is or is also present which preferably is or includes water.

Preferably or alternatively said liquid vehicle is or includes one compatible liquid organic compound, preferably selected from mineral and vegetable oils.

Preferably the ionophore antibiotic(s) has been milled in the presence of at least one of:

i) a compatible polyglycoside capable of acting as a dispersing agent,

ii) a compatible lignosulfonate capable of acting as a dispersing agent, and

iii) a compatible and suitable glycol in a form capable of acting as a dispersing agent.

Preferably the milling is in the absence of any suspension agent, or alternatively a suspension agent selected from the gums as previously described is present; preferably the suspension agent is one or more of xanthan gum, guar gum, acacia gum and a cellulose gum.

Preferably a liquid vehicle(s) was present at the time of milling of the ionophore antibiotic sufficient to reduce the consistency of the mill mix to a millable consistency.

According to a further aspect of the invention there is provided animal drinking water having at least one particulate ionophore antibiotic substantially uniformly suspended therein, wherein the ionophore antibiotic is stably suspended.

Preferably the ionophore antibiotic remains stably suspended for at least 24 days.

Preferably particles of the ionophore antibiotic(s) are of a mean particle size of less than 10 microns; more preferably they are of a mean particle size of substantially 5 microns.

Preferably the drinking water is made by a proportioned mix dispensing there into of a more concentrated aqueous suspension of the ionophore antibiotic and dispersing agents present in that more concentrated aqueous suspension provides the substantially uniform dispersion of the ionophore antibiotic(s) in the drinking water.

Preferably the more concentrated aqueous suspension contains ore or more suspension agents.

According to a further aspect of the invention there is provided animal drinking water having at least one particulate ionophore antibiotic substantially uniformly suspended therein, wherein the particles of the ionophore antibiotics are of a mean particle size less than 10 microns; more preferably the particle size of the ionophore antibiotics are of a mean particle size of substantially 5 microns.

Preferably the ionophore antibiotic remains stably suspended for at least 24 days.

According to a further aspect of the invention there is provided trough water accessible to an animal to drink, the water having a particulate ionophore antibiotic substantially uniformly suspended therein, wherein the ionophore antibiotic is stably suspended. Preferably the ionophore antibiotic remains stably suspended for at least 24 days. Preferably the particles of the ionophore antibiotic(s) is of a mean particle size of less than 10 microns; more preferably the particles of the ionophore antibiotic(s) is of a mean particle size of substantially 5 microns.

Preferably the trough water is made by a proportioned mix dispensing there into of a more concentrated aqueous suspension of the ionophore antibiotic and dispersing agents present in that more concentrated form provides the substantially uniform dispersion of the ionophore antibiotic(s) in the drinking water.

Preferably the more concentrated aqueous suspension contains ore or more suspension agents.

According to a further aspect of the invention there is provided trough water accessible to an animal to drink the water having a particulate ionophore antibiotic substantially uniformly suspended therein, wherein the ionophore antibiotic remains stably suspended for at least 24 days.

Preferably the particle size of the ionophore antibiotics are substantially 5 microns. Preferably the ionophore antibiotic remains stably suspended for at least 24 days.

According to a further aspect of the invention there is provided a method of dispensing a particulate ionophore antibiotic into a body of drinking water which comprises or includes the steps of:

taking a composition comprising or including at least one ionophore antibiotic of mean particle size of less than 20 microns and at least one dispersing agent:

forming an aqueous suspension of the composition that has the ionophore antibiotic(s) substantially uniformly dispersed therein and which aqueous suspension is available for dispensing into animal drinking water, and

dispensing at a rate (continuously or continually), that aqueous suspension into the drinking water or a makeup supply of water thereto so as to provide the body of drinking water with a uniform dispersion of the ionophore antibiotic(s) therein which is within an acceptable imbibing concentration range for the animal or animals having access thereto.

Preferably the mean particle size of the at least one ionophore antibiotic is substantially 5 microns.

Preferably said composition is in the form of a stable aqueous suspension prior to the forming of an aqueous suspension and the subsequent dispensing thereof into the drinking water or a make up supply of water.

Preferably said composition includes one or more dispersing agent selected from one or more of the following:

i) a compatible polyglycoside capable of acting as a dispersing agent,

ii) a compatible lignosulfonate capable of acting as a dispersing agent, and

iii) a compatible and suitable glycol in a form capable of acting as a dispersing agent.

Preferably a suspension agent selected from gums (as previously described) is present.

Preferably the ionophore antibiotic(s) remains stably suspended for at least 24 days.

According to a further aspect of the invention there is provided a body of drinking water having a particulate ionophore suspended therein prepared by the method as previously described.

According to a further aspect of the invention there is provided a method of forming a suspendable composition of at least one ionophore antibiotic and which suspendable composition is capable of subsequent dilution, said method including, prior to any optional presence of water and optional presence of a suspension agent, milling the ionophore antibiotic(s) with a suitable glycol.

Preferably said milling takes place in the presence of at least some liquid, which preferably includes water.

Preferably no gum is present.

Preferably said milling is to a mean particle size very much less than 20 microns; more preferably the mean particle size is of about 5 microns.

Preferably the milling takes place in the presence of a suitable lignosulfonate.

Preferably said milling takes place in the presence of a suitable Polyglycoside.

Preferably said milling takes place in the presence of a suitable antifoam agent.

According to a further aspect of the invention there is provided a suspendable composition of at least one ionophore antibiotic prepared according to the method as previously described.

According to a further aspect of the invention there is provided a method of forming a suspendable composition of at least one ionophore antibiotic and which suspendable composition is capable of subsequent dilution, said method including, prior to any optional presence of water and optional presence of a suspension agent, milling the ionophore antibiotic(s) with a suitable dispersion agent.

Preferably the milling takes place in the presence of a suitable lignosulfonate. Preferably or alternatively said milling takes place in the presence of a suitable polyglycoside.

Preferably said suitable dispersion agent is selected from the group consisting of a suitable lignosulfonate and a suitable polyglycoside.

Preferably the mean particle size is less than 20 microns; more preferably less than 10 microns; even more preferably the mean particle size is substantially 5 microns.

Preferably said milling takes place in the presence of a suitable antifoam agent.

Preferably said milling takes place in the presence of at least some liquid.

Preferably a suitable glycol is present.

Preferably no gum is present.

According to a further aspect of the invention there is provided a dose dispensable ionophore antibiotic composition comprising or including:

at least one ionophore antibiotic,

at least one dispersing agent,

at least one suspension agent, and

water,

wherein the ionophore antibiotic(s) and at least one dispersing agent have been milled together in the presence of a liquid (which may be water or may include water but not necessarily so) and where the dispersing agent has been added post-milling in the presence of water.

Preferably the ionophore antibiotic is of a mean particle size of less than 50 microns; more preferably a mean particle size of less than 20 microns.

Preferably at least one suspension agent is or includes a gum and no such gum was present at the milling procedure.

Preferably some water was present at the milling procedure.

Preferably a polyglycoside was present as a dispersing agent at the milling stage.

Preferably a lignosulfonate was present as a dispersing agent at the milling stage.

Preferably a glycol was present as a dispersing agent at the milling stage.

Preferably an alkyl polyglycoside, a lignosulfonate and a propylene glycol are present at the milling stage.

Preferably any one or more of an antifoam agent or system, a preservative, a debittering agent and a pH buffering system is present.

According to a further aspect of the invention there is provided a milled product useful in providing an aqueous suspension of at least one ionophore antibiotic, said product being the milled outcome of a mill mix of at least

at least one ionophore antibiotic, and

at least one dispersing agent,

wherein the mill mix has been substantially free of suspension agents selected from the gums previously described and the mill mix has included at least one liquid component (which optionally can be the or one of the dispersing agent(s) or an additional material),

and wherein, in the product, the process of milling has resulted in some physical association of the or at least one dispersing agent on the ionophore antibiotic particles,

and wherein, in the product, the mean particle size of the ionophore antibiotic(s) is less than 20 microns.

Preferably the mean particle size is substantially 5 microns.

According to a further aspect of the invention there is provide a suspendable composition for substantial dilution by water, said method comprising or including:

(I) milling at least one ionophore antibiotic and at least one dispersing agent so as to provide some physical association of the or at least one dispersing agent on ionophore antibiotic particles of a mean particle size of less than 20 microns, and

(II) blending the post mill ionophore antibiotic(s)/dispersing agent(s) milled outcome with at least a suspension agent.

This invention may also be said broadly to consist in the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.

The invention consists in the foregoing and also envisages constructions of which the following gives examples.

Definitions

Wherein the specification the following terms are used there are a number of possible alternatives, examples of which follow:

Surfactants/Dispersants:

sorbitan esters

ethoxylated sorbitan esters

castor oil ethoxylates

ethoxylated fatty acids

ethoxylated alcohols

polyethylene glycol fatty acids

condensed napthalene sulphonic acid

glycerol esters

phosphate esters

sodium lauryl sulphate

sodium polyacrylate

ammonium polyacrylate

octyl phenyl ethoxylates

nonyl phenyl ethoxylates

quaternary ammonium compounds

sulphosuccinates

soya lecithin

Suspending aids, including gums:

aluminium stearate

silicon dioxide

modified clay including Smectite, Monmorillonite, Hectorite, Bentonite

dehydrogenated castor oil

titanium chelates

alkali soluble acrylic polymers

nonionic diurethanes

polyvinyl pyrollidone

polyvinyl alcohol

guar gum

gum arabic

hydroxy propyl methyl cellulose

hydroxy ethyl cellulose

sodium carboxy methyl cellulose

hydrophobically modified hydroxy ethyl cellulose

Defoamers/Antifoams

polymethyl siloxane

unsaturated hydrocarbon oil

mineral oil

silica in oil type

Preservatives:

oxazolidines

benzothiazoles

benzalkonium chloride

substituted triazines

isothiazolones

hemiacetals

substituted benzoates

zinc pyridinethiol oxide

sodium pyridinethiol oxide

Buffering agents:

potassium tetroxalate

potassium hydrogen tartrate

potassium hydrogen phthalate

borax

### DETAILED DESCRIPTION OF THE INVENTION

Preferred forms of the present invention will now be described with reference to the accompanying drawings in which

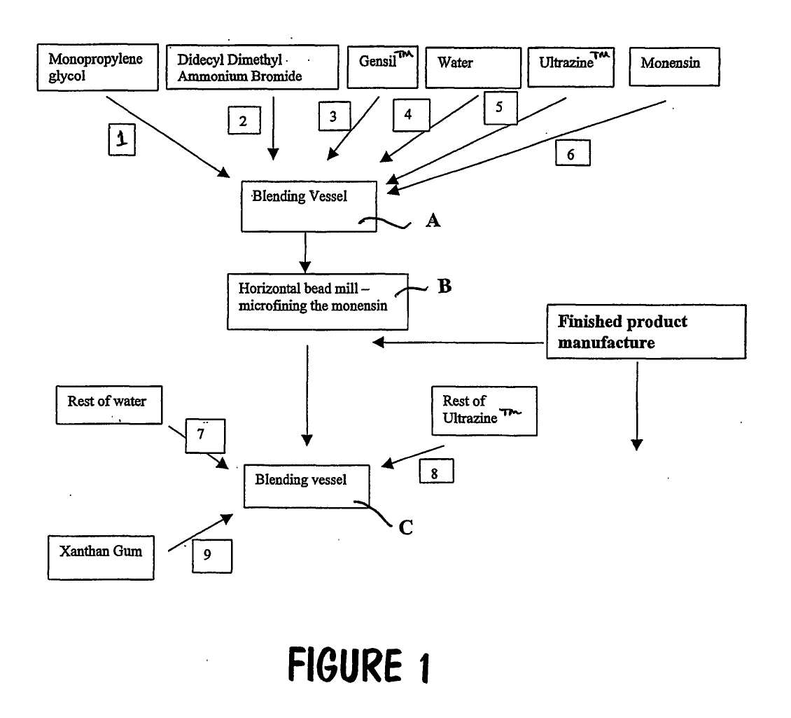

FIG. 1: shows one mixing procedure utilised in accordance with the invention,

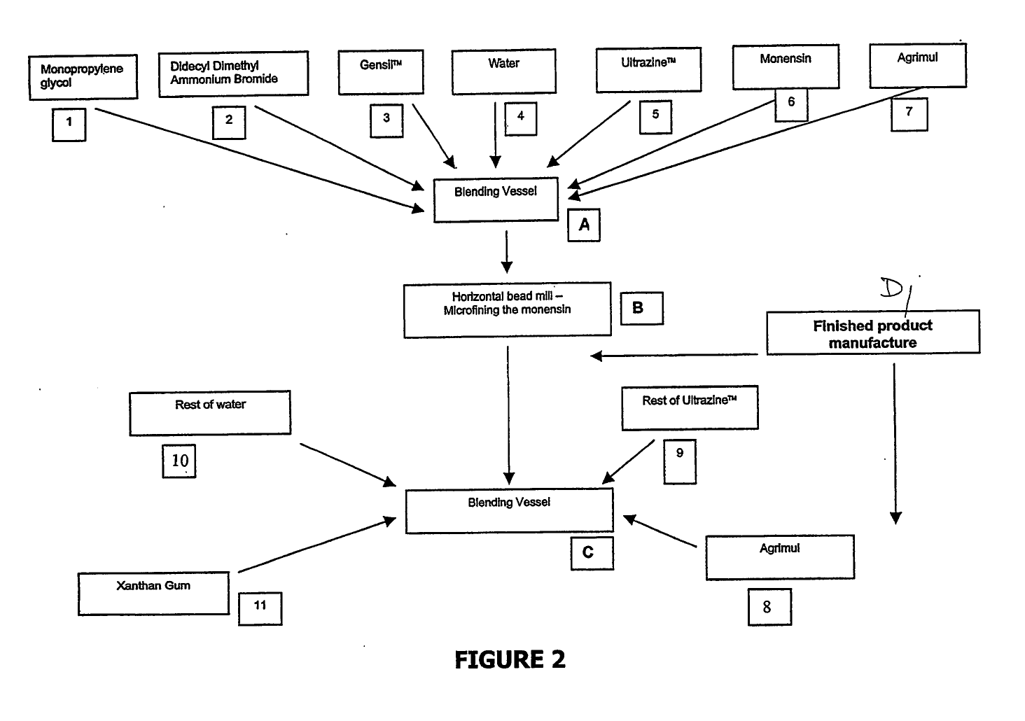

FIG. 2: shows a second mixing procedure utilised in accordance with the invention, and

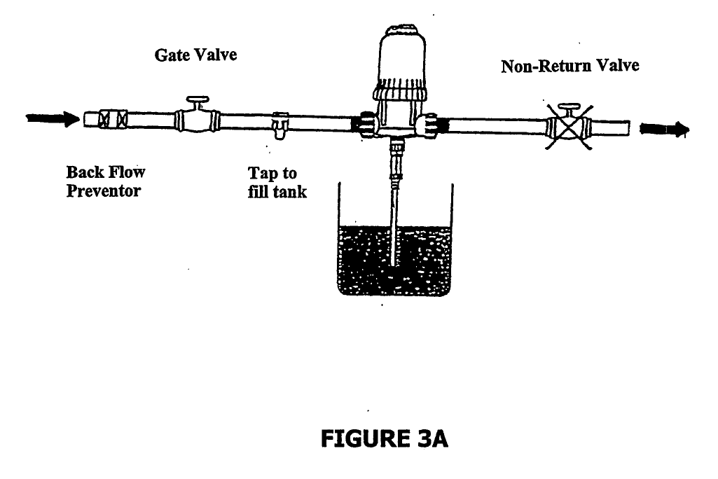

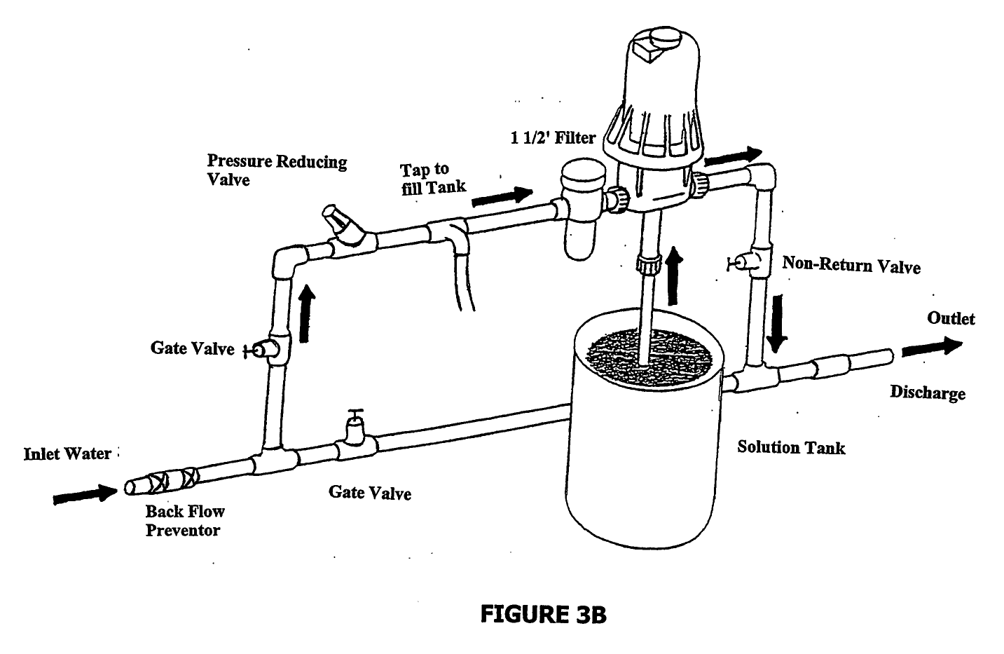

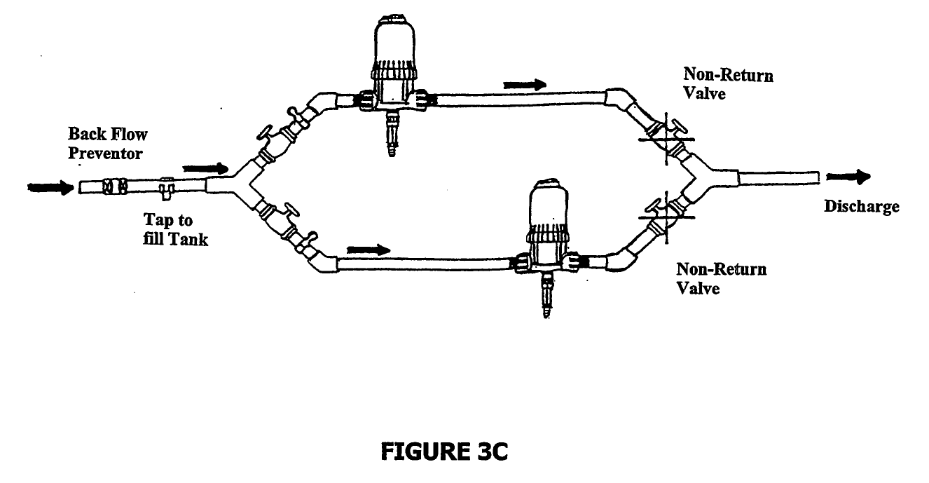

FIGS. 3A-3C: illustrate a DOSTATRON™ trough treatment system in three different modes of operation.

Shown in FIGS. 3A to 3C are three installations of a DOSATRON™ type installation used for dosing materials into drinking water. The DOSATRON™ apparatus is recommended as being usable in line (FIG. 3A), on a bypass line (FIG. 3B) and in parallel (FIG. 3C). In this respect see the publicity materials available from the New Zealand distributor Mark Bell-Booth Ltd.

The DOSATRON™ apparatus itself employees a dosing piston driven by a volumetric hydraulic piston motor. The spread rhythm of the motor is proportional to the flow of water passing through the system and thus the rate of injection will likewise remain proportional in its reciprocating motion.

The apparatus has the capability of a wide range of daily dose settings and the parallel arrangement shown in FIG. 3 allows these to be doubled owing to the use of two DOSATRON™ dispensing units.

Preferred forms will now be described with reference to the following examples of which the mixing procedures utilised is preferably as depicted and sequenced in one of FIG. 1 or 2.

The present invention recognises that having evolved a suitable stable aqueous suspension of microfine monensin a predictable in feed thereof to animals is possible simply by providing to the animal drinking water which includes the microfine ionophore antibiotic dispersed therein, said ionophore antibiotic having been included in the water supply by an intake directly or indirectly from a composition as previously defined. The less dilute the concentrate the more suspension agent we have found to be desirable.

Preferably the composition is for use in one or more of the trough treatment systems available.

### EXAMPLE 1

| Sodium Monensin | 6.33% (about 6% monensin) w/v | |
|---|---|---|
| Monopropylene glycol | 10% | w/v |
| ULTRAZINE NA ™ | 4-5% | w/v |
| Disodium Phosphate Anhydrous | 0.355% | w/v |
| MonoPotassium phosphate Dihydrate | 0.04% | w/v |
| Dialkyl dimethyl ammonium bromide | .0064% | w/v |
| Xanthan Gum | | |
| 0.4% w/v | | |
| Simethicone | ] GENSIL ™ | |
| 0.333% w/v | | |
| Silicon Dioxide] Antifoam | 0.167% | w/v |
| Water to 100% | | |

Milling has resulted in a mean particle size for sodium monensin of 5 microns.

### EXAMPLE 2

| Sodium Monensin | 6.33% (about 6% monensin) w/v | |
|---|---|---|
| Monopropylene glycol | 10% | w/v |
| Alkyl Polyglycoside | 0.5% | w/v |
| Disodium Phosphate Anhydrous | 0.355% | w/v |
| MonoPotassium phosphate Dihydrate | 0.04% | w/v |
| Dialkyl dimethyl ammonium bromide | .0064% | w/v |
| Xanthan Gum | 0.4% | w/v |
| Simethicone] GENSIL ™ | 0.333% | w/v |
| Silicon Dioxide] Antifoam | 0.167% | w/v |
| Water to 100% | | |

Milling has resulted in a mean particle size for sodium monensin of 5 microns.

### EXAMPLE 3

| Sodium Monensin | 6.33% (about 6% monensin) w/v | |
|---|---|---|
| Monopropylene glycol | 10% | w/v |
| ULTRAZINE NA ™ | 4-5% | w/v |
| Disodium Phosphate Anhydrous | 0.355% | w/v |
| MonoPotassium phosphate Dihydrate | 0.04% | w/v |
| Dialkyl dimethyl ammonium bromide | .0064% | w/v |
| Xanthan Gum | 0.4% | w/v |
| Simethicone] GENSIL ™ | 0.333% | w/v |
| Silicon Dioxide] Antifoam | 0.167% | w/v |
| Sorbitol as a Debittering agent | 3.5% | w/v |
| Water to 100% | | |

Milling has resulted in a mean particle size for sodium monensin of 5 microns.

### EXAMPLE 4

| Sodium Monensin | 6.33% (about 6% monensin) w/v | |
|---|---|---|
| Monopropylene glycol | 10% | w/v |
| Alkyl Polyglycoside | 0.5% | w/v |
| Disodium Phosphate Anhydrous | 0.355% | w/v |
| MonoPotassium phosphate Dihydrate | 0.04% | w/v |
| Dialkyl dimethyl ammonium bromide | .0064% | w/v |
| Xanthan Gum | 0.4% | w/v |
| Simethicone] GENSIL ™ | 0.333% | w/v |
| Silicon Dioxide] Antifoam | 0.167% | w/v |
| Sorbitol as a Debittering agent | 3.5% | w/v |
| Water to 100% | | |

Milling has resulted in a mean particle size for sodium monensin of 5 microns.

### EXAMPLE 5

| Ingredient | | | |
|---|---|---|---|
| name (common or | | | |
| chemical) | CAS number | Quantity (% w/w) | Function |
| Sodium Monensin | 17090-79-8 | 6.747% | Active |
| QA 166H | | | |
| Monopropylene | 57-55-6 | 10% | Antifreeze |
| glycol | | | |
| Didecyl dimethyl | 2390-68-3 | 0.1% | Preservative |
| Ammonium | | | |
| Bromide | | | |
| Xanthan Gum | 11138-66-2 | 0.5% | Suspension agent |
| Sodium ligno- | 8061-51-6 | 4.1% | Wetting agent |
| sulphonate | | | |
| Alkyl Polyglycoside | 68515-73-1 | 3.8% | Wetting agent |
| Simethicone | 8050-81-5 | 0.333% | Antifoam |
| Silicon Dioxide | 7631-86-9 | 0.167% | Antifoam |
| Water balance q.v | 7732-18-5 | 74.47% | diluent |

Milling has resulted in a mean particle size for sodium monensin of 5 microns.

Simethicone and Silicon dioxide together make up the proprietary brand “Gensil”.

1. Preparation Procedure

The formulations of each of Examples 1 to 4 can be prepared by the procedure shown in FIG. 1. The formulation of Example 5 is prepared by the procedure shown in FIG. 2. The preferred method of preparing a formulation such as Example 1 is as follows.

As can been seen from FIGS. 1 and 2 a blending vessel (A) which can, if desired, be the horizontal bead mill (B) but is preferably not, and a blending vessel (C) are utilised as the apparatus.

Most preferably however there is a three stage equipment base for the process viz. blending vessel (A), horizontal bead mill (B) for microfining the ionophore antibiotic and a blending vessel (C).

As can be seen from FIG. 1 ingredients 1 through 6 are blended in the reference number sequence in the blending vessel (A) prior to passage into the horizontal bead mill (B). These pre-blended materials include:

monopropylene glycol,

dialkyl dimethyl ammonium bromide,

GENSIL™ antifoam,

some water,

ULTRAZINE NA™ wetting agent, and

monensin.

After the milling that premill mix, the product can be taken away either as an intermediate product (eg; post mill product) for subsequent use elsewhere for blending. Preferably however the output milled mix passes to blending vessel (C) where it is blended with the rest of the water (7), the remainder of the ULTRAZINE NA™ wetting agent (8) and the xanthan gum or other dispersion agent (9).

With a formulation whether to the formula of Example 1 or Example 3 or another (FIGS. 1 and 2 do not refer to the buffering system nor to a debittering agent) very good suspensibility is obtained both of the concentrate and of a subsequent diluted form (eg; in a trough usage where the dilution is, for example, to about 3 to 6 ppm monensin).

The numerals 1-9 (FIG. 1) or 1-11 (FIG. 2) indicate the preferred sequence of ingredient addition. With reference to FIG. 1, a pre-mill non sequenced mix of components 1 through 6 in the blending vessel (A) will still lead to a good mill mix yet is detrimental to the best suspensibility of the diluted form.

A preferred formulation as in Example 1 made by a procedure as in FIG. 1 has a capability of being added as an aqueous concentrate into a large volume of water such as might be experienced in providing an infeed into a water system.

It is important to note the following processing issues:

the order of addition of the components to the grind base premix is not critical

the appropriate particle size induced by the grinding operation is critical

the order of addition of the components in the makeup tank is critical.

2. The Mill Mix

The mill mix is an important factor in the invention. It is the pre-mix of components which are added into the bead mill. The composition (identity and amount) is important in determining the ultimate particle size and the coatings on the particles which result. The relative quantities (eg of MPG: monensin) are important in this respect.

3. Stability Data

a) Shelf Life Stability

The following shelf file stability data indicates the 6% concentrate exhibits stability, at differing temperatures for at least 3 months (the length of time of the trials).

Trough Treatment 6% Concentrate Shelf Life Study

Batch number: I183

| | Time 0 | 1 months | 2 months | 3 months |
|---|---|---|---|---|
| Appearance | Normal | Normal | Normal | Normal |
| 25° C. Sample | | | | |
| Coliforms | <1 | <1 | <1 | <1 |
| Monensin Biopotency | 5.7 | 5.5 | 5.4 | 5.6 |
| PH | 8.11 | 8.00 | 7.77 | 7.51 |
| Yeasts and Moulds | <1 | <1 | <1 | <1 |
| APC | <10 | <10 | <10 | <10 |
| 42° C. Sample: | | | | |
| Coliforms | <1 | <1 | <1 | <1 |
| Monensin Biopotency | 5.7 | 5.6 | 5.4 | 5.4 |
| PH | 8.11 | 7.60 | 7.39 | 7.28 |
| Yeasts and Moulds | <1 | <1 | <1 | <1 |
| APC | <10 | <10 | <10 | <10 |

*Appearance—normal means light brown gelatinous liquid with brown specks present.

b) Positional Stability

The following experiments detail the positional stability of the trough treatment of the invention (TT).

Positional stability studies were conducted with a DOSATRON™ trough treatment system. This is a proportional feed system which delivers a % mix in proportion to demand. Such an administration system is able to spread delivery of a complete batch over 24 hours.

FIG. 3 illustrates a dosation system used in obtaining the positional stability data

### EXAMPLE 1

Trough Treatment (TT) (600 ppm) in a 200L plastic solution tank with a functioning Dosatron 8000.

Method:

100 litres water were added to the solution tank connected to a Dosatron 8000.

2 litres TT were mixed well with 2 litres water.

This mix was added to the half-full solution tank, filled to the 200 litre level, mixed thoroughly.

The Dosatron was set at 2% and the water flow at 400 litres/hour to ensure the solution tank is completely used within 24 hours.

The draw off tube from the Dosatron was set at a height 10 cm from the bottom of the tank.

Sampling

Samples for assay were taken from the draw-off taps. Before sampling, a 50 ml sample was drawn off and discarded. 1×100 ml samples are taken from each of the 4 taps set at either the top, middle or bottom of the tank. Samples will be taken at the specified time intervals.

Each sample taken was individually identified and 50 ml from each sample taken and to a pooled sample (total Vol 200 ml).

This sample was that assayed and the other samples retained.

Samples were also taken from the four-tap set, here positioned in the plastic tank at a level 10 cm from the bottom of the tank.

Samples were taken 0, 12, 24 hours after mixing.

After 24 hours a further 4×100 ml samples were taken from the bottom of the tank. Each sample taken was individually identified and 50 ml from each sample taken and pooled (total Vol 200 ml). This sample was assayed and the other samples retained. In this case the drench gun tube attached to the rod was used.

### EXAMPLE 2

Measurement of the positional stability of the TT (3000 ppm) in a static 200 L plastic solution tank over a 4 day period

Method

Before filling the tank, the bottom tap hoses were bent so the end was 5 cm from the bottom of the tank.

100 litres of water was added to the solution tank.

10 litres of TT was mixed well with 10 litres water.

This mix was added to the half-full solution tank, filled to the 200 litre level and mixed.

Sampling

Samples for assay were taken from the draw-off taps. Before sampling, a 50 ml sample was drawn off and discarded. Each sample taken was individually identified and 50 ml from each sample taken and pooled (total Vol 200 ml). This sample was that assayed and the other samples retained.

Samples were taken from the top, middle and bottom of the tank at the intervals of 0, 12 hrs, 24 hrs, 2 days and 4 days after mixing.

### EXAMPLE 3

Measurement of the positional stability of TT (6 ppm) in a concrete trough over a 24 day period.

Method

50 litres of water was added to the solution tank connected to the Dosatron 8000. 1 litre of TF was mixed well with 1 litre water.

This mix was added to the half-full solution tank, filled to the 100 litre level and mixed.

Set the Dosatron at 1% to ensure a trough concentration of 6 ppm.

The trough was connected to the solution tank containing the Dosatron and TT.

The new concrete trough was scrubbed clean and the water in the trough pH tested before use.

A neutral pH (7-8) is acceptable.

The water was discarded before filling from the Dosatron 8000

Activation of the ball-cock filled the trough with TT (6 ppm).

After filling the trough herd drinking was simulated by siphoning water from the top of the trough to activate the ball-cock. 5000 litres was siphoned from the trough over a 24 hour period

Sampling

The draw-off tube from the drench gun was attached to a rigid pole. The end of the draw off tube was set to sample the trough from 3 levels—top, middle and bottom. 4×100 ml samples was taken from each position.

Each sample taken was individually identified and 50 ml from each sample taken and pooled (total Vol 200 ml).

This sample was that assayed and the other samples retained.

Sampling of the trough at the intervals of 0, 6 hrs, 12 hrs, 24 hrs, 5 days, 10 days and 24 days after mixing.

### EXAMPLE 4

Measurement of the amount of monensin settling in the drum after mixing TT at 3000 ppm and leaving for 4 days undisturbed.

Methods

100 litres water was added to the solution tank connected to the Dosatron 8000. 10 litres of TT was mixed with 10 litres of water.

This mix was added to the half-full solution tank, filled to the 200 litre level and mixed.

Sampling

Samples for assay were taken from the bottom of the tank.

Before sampling, opened all middle taps and bottom taps to provide a slow release of tank mix. The rate of removal of the fluid was slow enough so the bottom of the tank was not disturbed.

After the level of water fell below the bottom taps, mixed the bottom of the tank thoroughly and drew off 4×100 ml samples taken at different locations.

Each sample taken was individually identified and 50 ml from each of the samples taken and pooled (total Vol 200 ml).

The sample was that assayed and the other samples retained.

N. B Please calculate the volume of water left in the drum before taking the samples. This will be to calculate the total amount of monensin settling in the bottom of the tank after 4 days

Positional Stability Results and Discussion

The test had the following accuracy parameters:

Repeatability. The difference between results of duplicate portions of the same sample tested in the same run should not exceed 10% of the mean result. Recent results indicate that duplicate results are not exceeding 5.4% of the mean result.

Reproducibility: The difference between results of portions of the same sample tested at different times by different analysts should not exceed 15% of the mean result.

The repeatability and reproducibility data affords us quantifiable parameters for substantial homogeneity, indicating a substantially uniform suspension over time.

TABLE 1
Example 1 Results (600 ppm)

| Time | Bottom Sample Result |
|---|---|
| 0 hours | 556 ppm |
| 12 hours | 504 ppm |
| 24 hours | 439 ppm |

TABLE 2
Example 1 Repeated Trial Results (600 ppm)

| Time | Bottom Sample Result |
|---|---|
| 0 hours | 580 ppm |
| 12 hours | 470 ppm |
| 24 hours | 420 ppm |

TABLE 3
Example 2 Results (3000 ppm)

| | Top Sample | Middle Sample | Bottom Sample |
|---|---|---|---|
| Time | (ppm) | (ppm) | (ppm) |
| 0 hours | 2970 | 2900 | 3030 |
| 12 hours | 2680 | 2720 | 2760 |
| 24 hours | 2930 | 2970 | 3060 |
| 2 days | 2960 | 2950 | 3030 |
| 4 days | 2190 | 2930 | 3050 |

TABLE 4
Example 3 Results (6 ppm)

| | Top Sample | Middle Sample | Bottom Sample |
|---|---|---|---|
| Time | (ppm) | (ppm) | (ppm) |
| 0 hours | 5.0 | 5.5 | 5.1 |
| 6 hours | 4.9 | 4.6 | 5.2 |
| 12 hours | 4.7 | 4.9 | 5.0 |
| 24 hours | 5.6 | 5.2 | 5.0 |
| 4 days | 5.1 | 5.3 | 4.3 |
| 10 days | 5.5 | 5.1 | 5.8 |
| 24 days | 5.0 | 5.0 | 5.3 |

1. An ionophore antibiotic composition capable of dilution with water to a substantially stable dispersed form in all water then present, said composition comprising or including:
at least one ionophore antibiotic of a mean particle size of less than 20 microns,
and at least one; dispersing agent.

2. A composition as claimed in claim 1 wherein the mean particle size of at least one ionophore antibiotic is substantially 5 microns.

3. A composition as claimed in claim 1 or 2 wherein the dispensed form in water remains substantially homogeneous for at least 24 days.

4. A composition as claimed in any one of claims 1 to 3 wherein said at least one dispersing agent is selected from one or more of the following:
iv) a compatible polyglycoside capable of acting as a dispersing agent,
v) a compatible lignosulfonate capable of acting as a dispersing agent, and
vi) a compatible and suitable glycol in a form capable of acting as a dispersing agent.

5. A composition as claimed in claim 3 or 4 wherein a liquid vehicle is or is also present.

6. A composition as claimed in claim 5 wherein said liquid vehicle is or includes water.

7. A composition as claimed in claim 5 or 6 wherein said liquid vehicle is or includes one compatible liquid organic compound.

8. A composition as claimed in claim 7 wherein said organic compound is selected from mineral and vegetable oils.

9. A composition as claimed in any one of claims 1 to 8 wherein said ionophore antibiotic(s) has been milled in the presence of at least one of:
iv) a compatible polyglycoside capable of acting as a dispersing agent,
v) a compatible lignosulfonate capable of acting as a dispersing agent, and
vi) a compatible and suitable glycol in a form capable of acting as a dispersing agent.

10. A composition as claimed in claim 9 wherein the milling is in the absence of any suspension agent.

11. A composition as claimed in claim 9 wherein a suspension agent selected from the gums as previously described is present.

12. A composition as claimed in claim 11 wherein the suspension agent is one or more of xanthan gum, guar gum, acacia gum and a cellulose gum.

13. A composition as claimed in any one of claims 9 to 11 wherein a liquid vehicle(s) was present at the time of milling of the ionophore antibiotic sufficient to reduce the consistency of the mill mix to a millable consistency.

14. Animal drinking water having at least one particulate ionophore antibiotic substantially uniformly suspended therein, wherein the ionophore antibiotic is stably suspended.

15. Drinking water as claimed in claim 14 wherein the ionophore antibiotic remains stably suspended for at least 24 days.

16. Drinking water as claimed in claim 14 or 15 wherein the particles of the ionophore antibiotic(s) are of a mean particle size of less than 10 microns.

17. Drinking water as claimed in claim 16 wherein the particles of the ionophore antibiotic(s) are of a mean particle size of substantially 5 microns.

18. Drinking water as claimed in 17 it being made by a proportioned mix dispensing there into of a more concentrated aqueous suspension of the ionophore antibiotic and dispersing agents present in that more concentrated aqueous suspension provides the substantially uniform dispersion of the ionophore antibiotic(s) in the drinking water.

19. Drinking water as claimed in claim 18 which the more concentrated aqueous suspension contains ore or more suspension agents.

20. Animal drinking water having at least one particulate ionophore antibiotic substantially uniformly suspended therein, wherein the particles of the ionophore antibiotics are of a mean particle size less than 10 microns.

21. Animal drinking water as claimed in claim 20 wherein the particle size of the ionophore antibiotics are of a mean particle size of substantially 5 microns.

22. Animal drinking water as claimed in claim 21 wherein the ionophore antibiotic remains stably suspended for at least 24 days.

23. Trough water accessible to an animal to drink, the water having a particulate ionophore antibiotic substantially uniformly suspended therein, wherein the ionophore antibiotic is stably suspended.

24. Trough water as claimed in claim 23 wherein the ionophore antibiotic remains stably suspended for at least 24 days.

25. Trough water as claimed in claim 24 wherein the particles of the ionophore antibiotic(s) is of a mean particle size of less than 10 microns.

26. Trough water as claimed in claim 25 wherein the particles of the ionophore antibiotic(s) is of a mean particle size of substantially 5 microns.

27. Trough water as claimed in 26 it is being made by a proportioned mix dispensing there into of a more concentrated aqueous suspension of the ionophore antibiotic and dispersing agents present in that more concentrated form provides the substantially uniform dispersion of the ionophore antibiotic(s) in the drinking water.

28. Drinking water as claimed in claim 27 which the more concentrated aqueous suspension contains ore or more suspension agents.

29. Trough water accessible to an animal to drink the water having a particulate ionophore antibiotic substantially uniformly suspended therein, wherein the ionophore antibiotic remains stably suspended for at least 24 days.

30. Animal drinking water as claimed in claim 29 wherein the particle size of the ionophore antibiotics are substantially 5 microns.

31. Animal drinking water as claimed in claim 30 wherein the ionophore antibiotic remains stably suspended for at least 24 days.

32. A method of dispensing a particulate ionophore antibiotic into a body of drinking water which comprises or includes the steps of:
taking a composition comprising or including at least one ionophore antibiotic of mean particle size of less than 20 microns and at least one dispersing agent:
forming an aqueous suspension of the composition that has the ionophore antibiotic(s) substantially uniformly dispersed therein and which aqueous suspension is available for dispensing into animal drinking water, and
dispensing at a rate (continuously or continually), that aqueous suspension into the drinking water or a makeup supply of water thereto so as to provide the body of drinking water with a uniform dispersion of the ionophore antibiotic(s) therein which is within an acceptable imbibing concentration range for the animal or animals having access thereto.

33. A method as claimed in claim 32 wherein the mean particle size of the at least one ionophore antibiotic is substantially 5 microns.

34. A method of claim 32 or 33 wherein said composition is in the form of a stable aqueous suspension prior to the forming of an aqueous suspension and the subsequent dispensing thereof into the drinking water or a make up supply of water.

35. A method of claim 34 wherein said composition includes one or more dispersing agent selected from one or more of the following:
i) a compatible polyglycoside capable of acting as a dispersing agent,
iv) a compatible lignosulfonate capable of acting as a dispersing agent, and
v) a compatible and suitable glycol in a form capable of acting as a dispersing agent.

36. A method as claimed in claim 35 wherein a suspension agent selected from gums (as previously described) is present.

37. A method as claimed in any one of claims 32 to 36 wherein the ionophore antibiotic(s) remains stably suspended for at least 24 days.

38. A body of drinking water having a particulate ionophore suspended therein prepared by the method of anyone of claims 32 to 37.

39. A method of forming a suspendable composition of at least one ionophore antibiotic and which suspendable composition is capable of subsequent dilution, said method including, prior to any optional presence of water and optional presence of a suspension agent, milling the ionophore antibiotic(s) with a suitable glycol.

40. A method as claimed in claim 39 wherein said milling takes place in the presence of at least some liquid.

41. A method as claimed in claim 40 wherein said at least some liquid includes water.

42. A method as claimed in claim 41 wherein no gum is present.

43. A method as claimed in 41 or 42 wherein said milling is to a mean particle size very much less than 20 microns.

44. A method of 43 wherein the mean particle size is of about 5 microns.

45. A method of 44 wherein the milling takes place in the presence of a suitable lignosulfonate.

46. A method of 45 wherein said milling takes place in the presence of a suitable Polyglycoside.

47. A method of 46 wherein said milling takes place in the presence of a suitable antifoam agent.

48. A suspendable composition of at least one ionophore antibiotic prepared according to the method of any one of claims 39 to 47.

49. A method of forming a suspendable composition of at least one ionophore antibiotic and which suspendable composition is capable of subsequent dilution, said method including, prior to any optional presence of water and optional presence of a suspension agent, milling the ionophore antibiotic(s) with a suitable dispersion agent.

50. A method of 49 wherein the milling takes place in the presence of a suitable lignosulfonate.

51. A method of 50 wherein said milling takes place in the presence of a suitable polyglycoside.

52. A method as claimed in 51 wherein said suitable dispersion agent is selected from the group consisting of a suitable lignosulfonate and a suitable polyglycoside.

53. A method as claimed in 52 wherein the mean particle size is less than 20 microns.

54. A method of 53 wherein the mean particle size is less than 10 microns.

55. A method of 54 wherein the mean particle size is substantially 5 microns.

56. A method of 55 wherein said milling takes place in the presence of a suitable antifoam agent.

57. A method of 56 wherein said milling takes place in the presence of at least some liquid.

58. A method as claimed in claim 57 wherein a suitable glycol is present.

59. A method as claimed in claim 58 wherein no gum is present.

60. A dose dispensable ionophore antibiotic composition comprising or including:
at least one ionophore antibiotic,
at least one dispersing agent,
at least one suspension agent, and
water,
wherein the ionophore antibiotic(s) and at least one dispersing agent have been milled together in the presence of a liquid (which may be water or may include water but not necessarily so) and where the dispersing agent has been added post-milling in the presence of water.

61. A composition of claim 60 wherein the ionophore antibiotic is of a mean particle size of less than 50 microns

62. A composition of claim 61 wherein said ionophore antibiotic(s) is present at a mean particle size of less than 20 microns.

63. A composition of claim 62 wherein said at least one suspension agent is or includes a gum and no such gum was present at the milling procedure.

64. A composition as claimed in claim 63 wherein some water was present at the milling procedure.

65. A composition as claimed in claim 64 wherein a polyglycoside was present as a dispersing agent at the milling stage.

66. A composition as claimed in claim 64 wherein a lignosulfonate was present as a dispersing agent at the milling stage.

67. A composition as claimed in claim 64 wherein a glycol was present as a dispersing agent at the milling stage.

68. A composition as claimed in claim 64 wherein an alkyl polyglycoside, a lignosulfonate and a propylene glycol are present at the milling stage.

69. A composition as claimed in any one of claims 64-68 wherein any one or more of an antifoam agent or system, a preservative, a debittering agent and a pH buffering system is present.

70. A milled product useful in providing an aqueous suspension of at least one ionophore antibiotic, said product being the milled outcome of a mill mix of at least
at least one ionophore antibiotic, and
at least one dispersing agent,
wherein the mill mix has been substantially free of suspension agents selected from the gums previously described and the mill mix has included at least one liquid component (which optionally can be the or one of the dispersing agent(s) or an additional material),
and wherein, in the product, the process of milling has resulted in some physical association of the or at least one dispersing agent on the ionophore antibiotic particles,
and wherein, in the product, the mean particle size of the ionophore antibiotic(s) is less than 20 microns.

71. A milled product as claimed in claim 70 wherein the mean particle size is substantially 5 microns.

72. A method of preparing a suspendable composition for substantial dilution by water, said method comprising or including:
(I) milling at least one ionophore antibiotic and at least one dispersing agent so as to provide some physical association of the or at least one dispersing agent on ionophore antibiotic particles of a mean particle size of less than 20 microns, and (III) blending the post mill ionophore antibiotic(s)/dispersing agent(s) milled outcome with at least a suspension agent.
